(19) United States
(12) Patent Application Publication
(10) Pub. No.: US 2023/0363880 A1
(43) Pub. Date: Nov. 16, 2023

(54) Surgical Implant for Supporting Tissue Following Pelvic Organ Prolapse

(71) Applicants: Rebecca U. Ruefer, Bozeman, MT (US); Beda B.G. Ruefer, Bozeman, MT (US)

(72) Inventors: Rebecca U. Ruefer, Bozeman, MT (US); Beda B.G. Ruefer, Bozeman, MT (US)

(21) Appl. No.: 18/144,950

(22) Filed: May 9, 2023

Related U.S. Application Data
(60) Provisional application No. 63/342,246, filed on May 16, 2022.

(51) Int. Cl. A61F 2/00; A61L 31/06

(52) U.S. Cl. CPC A61F 2/0063; A61L 31/06; A61F 2002/0081; A61F 2240/001

(57) ABSTRACT

Disclosed is a process of making an expanded fluoropolymer, e.g., PTFE, article. The article is formed from a PTFE resin, calendered, and then expanded (e.g., multiaxially) at an elevated temperature. After cooling, the article is used to form a surgically implanted device, e.g., a surgical prosthetic patch. The article may be used in certain surgical procedures, such as those concerning pelvic organ prolapse.

### CROSS-REFERENCE TO RELATED APPLICATIONS

This application claims priority to U.S. application Ser. No. 63/342,246, filed May 16, 2022, which is incorporated by reference in its entirety herein.

### BACKGROUND OF THE INVENTION

### 1. Field

The disclosed embodiments relate generally to the field of prosthetic medical devices. More specifically, the field relates to the development of prosthetic medical devices implemented for supporting internal body structures, e.g., following Pelvic Organ Prolapse (POP).

### 2. Description of the Related Art

Pubovaginal patch procedures are very prevalently used to offer support needed to stabilize a patient's organ or other tissue extending out through a compromising aperture formed through another retaining tissue (e.g., muscle). The most common devices used in executing such processes are constructed of a nonabsorbable polypropylene mesh material, the ends of which can be anchored elsewhere in the patient's body.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages will be apparent from the following detailed description of the embodiments and the accompanying drawing figures.

Embodiments of the present invention include a process of making a surgical implant configured to provide support for a tissue following pelvic organ prolapse, the process comprises selecting an amount of a processed fluoropolymer; creating a paste by combining the amount of the processed fluoropolymer with an extrusion aid; compressing the paste into a pre-extrusion form to create a material; extruding the material to create an article that is substantially flat; calendaring the article to a desired thickness; drying the article by applying heat to the article at a first temperature; expanding the article in one or more directions; heat treating the article while the article is restrained in an expanded state to lock a structure of the article in place; and allowing the article to cool to result in an expanded fluoropolymer article; manipulating the expanded fluoropolymer article into a patch body, the patch body configured for use as the surgical implant.

In some aspects, the techniques described herein relate to a process, wherein manipulating the expanded fluoropolymer article into the patch body comprises cutting the expanded fluoropolymer into a pre-selected shape.

In some aspects, the techniques described herein relate to a process, wherein manipulating the expanded fluoropolymer article into the patch body comprises forming a substantially flat elongated body that extends from a first end to a second end.

In some aspects, the techniques described herein relate to a process, wherein manipulating the expanded fluoropolymer article into the patch body comprises connecting a first placement aid to the first end and a second placement aid to the second end, the first and second placement aids configured to aid in surgical placement of the patch body.

In some aspects, the techniques described herein relate to a process, wherein manipulating the expanded fluoropolymer article into the patch body comprises forming a substantially flat body with one or more placement aids connected to the substantially flat body at one or more connection locations.

In some aspects, the techniques described herein relate to a process, wherein manipulating the expanded fluoropolymer article into the patch body comprises forming a substantially flat elongated body having a divergence point wherein the substantially flat elongated body splits into a first prong and a second prong.

In some aspects, the techniques described herein relate to a process, wherein the first temperature is above a boiling point of the extrusion aid and below a sintering and coalescing temperature of the processed fluoropolymer.

In some aspects, the techniques described herein relate to a process, wherein expanding the article in one or more directions comprises multiaxial expanding of the article such that a resulting expanded article includes a plurality of multidirectional elongated fibrils extending between a plurality of nodes such that a plurality of pores are created between the plurality of nodes.

In some aspects, the techniques described herein relate to a process, wherein each of the plurality of multidirectional elongated fibrils has a length between approximately 0.5 microns to approximately 3.0 microns.

In some aspects, the techniques described herein relate to a process, wherein each of the plurality of multidirectional elongated fibrils has a length between approximately 0.5 microns to approximately 1.0 micron.

In some aspects, the techniques described herein relate to a process, wherein each of the plurality of pores has a size of less than or equal to 3.0 microns by 1.0 micron.

In some aspects, the techniques described herein relate to a process, wherein each of the plurality pores has a size of less than or equal to 2.0 microns.

In some aspects, the techniques described herein relate to a process, wherein the plurality of nodes form approximately 80% of a total surface area of the resulting expanded article.

Other embodiments of the present invention include a surgical implant configured to provide support for a tissue following pelvic organ prolapse, the surgical implant comprising a patch body formed from an expanded fluoropolymer article; wherein the expanded fluoropolymer article comprises a plurality of nodes and a plurality of elongated fibrils, the plurality of elongated fibrils interconnecting the plurality of nodes and forming a plurality of pores between the plurality of nodes.

In some aspects, the techniques described herein relate to a surgical implant, wherein each of the plurality of multidirectional elongated fibrils has a length between approximately 0.5 microns to approximately 3.0 microns.

In some aspects, the techniques described herein relate to a surgical implant, wherein each of the plurality of multidirectional elongated fibrils has a length between approximately 0.5 microns to approximately 1.0 micron.

In some aspects, the techniques described herein relate to a surgical implant, wherein each of the plurality of pores has a size of less than or equal to 3.0 microns by 1.0 micron.

In some aspects, the techniques described herein relate to a surgical implant, wherein each of the plurality of pores has a size of less than or equal to 2.0 microns

In some aspects, the techniques described herein relate to a surgical implant, wherein the plurality of nodes form approximately 80% of a total surface area of the patch body.

In some aspects, the techniques described herein relate to a surgical implant, wherein each of the plurality of nodes has a density of between approximately 2.0 grams to approximately 2.2 grams per cubic centimeter.

In some aspects, the techniques described herein relate to a surgical implant, wherein the expanded fluoropolymer article has a density of less than approximately 2.0 grams per cubic centimeter.

### BRIEF DESCRIPTION OF THE SEVERAL VIEWS OF THE DRAWINGS

Illustrative embodiments are described in detail below with reference to the attached drawing figures, which are incorporated by reference herein and wherein:

FIG. 1 illustrates a flowchart of a process of manufacturing a an expanded fluoropolymer according to the present disclosure;

FIG. 2 illustrates a micrograph taken of the material manufactured according to the process steps expressed herein where a multiaxial expansion is executed;

FIG. 3 depicts an anterior view of an article of a first embodiment;

FIG. 4 depicts a transverse view of an article of a second embodiment; and

FIG. 5 depicts an overall view of an article of a third embodiment.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the technology can include a variety of combinations and/or integrations of the embodiments described herein.

Synthetic meshes have been available for implant procedures for many decades and utilized for POP. The most common form of POP patch is constructed of polypropylene mesh, which is typically comprised of woven or knit filaments. A polypropylene mesh presents open structure which allows bacteria to penetrate. This can lead to post-operative infection. Although these devices have been implanted in many thousands of patients, there remain many post-implantation problems that have yet to be resolved. Most of these problems are clinically significant, and can end with surgical retrieval of the devices, which can lead to internal tissue damage.

One problem with these prior art polypropylene mesh patches is damage that can be caused upon surgical implant. Those skilled in the art will recognize that during surgery the polypropylene mesh patch is worked through bodily tissues, e.g., being pushed or pulled therethrough using needles or other devices. The edges of these prior art patches may be rough, and when passed through the body during surgical implant can create tissue damage, e.g., scar tissue, etc.

Any limited damage created upon implant of the polypropylene patch is not necessarily considered a bad thing, in that the damage causes an immediate inflammatory response, which ultimately helps incorporate the device structurally. But this damage can compromise tissues in undesirable ways also.

Another problem is that the polypropylene mesh degrades over time. This degradation can result in migration of patch segments or filaments, or even extrusion of the same. These obvious failures create harm, and often result in the need for removal of an already-implanted patch by a surgery far more complicated and potentially harmful than the initial implant procedure, and can leave behind massive scar tissue, as well as result in chronic pain moving forward.

Another problem is that of infection. The vulnerability of the polypropylene to infection is due in large part to the nature of the mesh material. Following implantation of a conventional polypropylene mesh patch, it is considered desirable that the surrounding tissues grow into the device. This ingrowth is seen as necessary to secure the polypropylene device in place. But this also makes the device extremely difficult to remove surgically, e.g., in the case of complications such as infection or structural degradation over time. Many times, surgery to remove the patch creates serious problems to the tissues on or around the implant.

The invention disclosed herein is a prosthetic medical patch and method of manufacturing said prosthetic medical patch, the patch made of a synthetic material. In embodiments, the synthetic material is a relatively closed expanded Polytetrafluoroethylene structure. The inventive design of the medical device provides for a strong, supportive, biocompatible patch that resists bacterial invasion and disintegration. It should be noted that the term “patch” as used herein means simply that the thing provides support in some way. Thus, the term would include any of the more specific properties of supporting (e.g., as a supportive membrane), providing a barrier, or containing against weakness existing in body tissues.

The prosthetic patch embodiments disclosed herein overcome the aforementioned problems significantly.

More specifically, the embodiments described herein comprise a novel synthetic prosthetic medical patch that is biocompatible, resists bacterial infection, exhibits significant long-term strength to support tissues, does not promote massive scar tissue, and will not deteriorate over time. Further, the patch embodiments exhibit strength adequate for intended use, are supple and tissue compliant to minimize scar tissue formation, and are durable, as PTFE, a fluoropolymer, is known to be resistant to biodegradation. The embodiments further comprise a process of manufacturing the synthetic prosthetic medical patch such that the patch embodies the features discussed above.

Embodiments are comprised of a processed fluoropolymer, such as Polytetrafluoroethylene (PTFE). Embodiments disclosed in the figures herein incorporate expanded PTFE, or “ePTFE” which is formed by expansion under heat. An ePTFE article can be manufactured to have fibril lengths such that the article is a relatively closed structure to prevent the infiltration of bacteria, yet supple for tissue compliance, and adequately strong for its intended use as a tissue support device. And of course, as ePTFE, it is fully biocompatible and will not degrade.

The microporous structure of known ePTFE articles is characterized by a plurality of nodes that are connected together by a plurality of fibrils. The nodes are essentially solid PTFE, having a density of between about 2.0 grams to about 2.2 grams per cubic centimeter, whereas the density of the expanded material is less than about 2.0 grams per cubic centimeter. The shape, size, and orientation of the nodes and fibrils within the structure can be controlled by varying the expansion rate, expansion ratio, number of expansion axes, and other processing parameters to yield many different structures. It is also known that properties such as the expandability and microstructure of the expanded article vary with the molecular weight, particle size, and other physical characteristics of the PTFE resin.

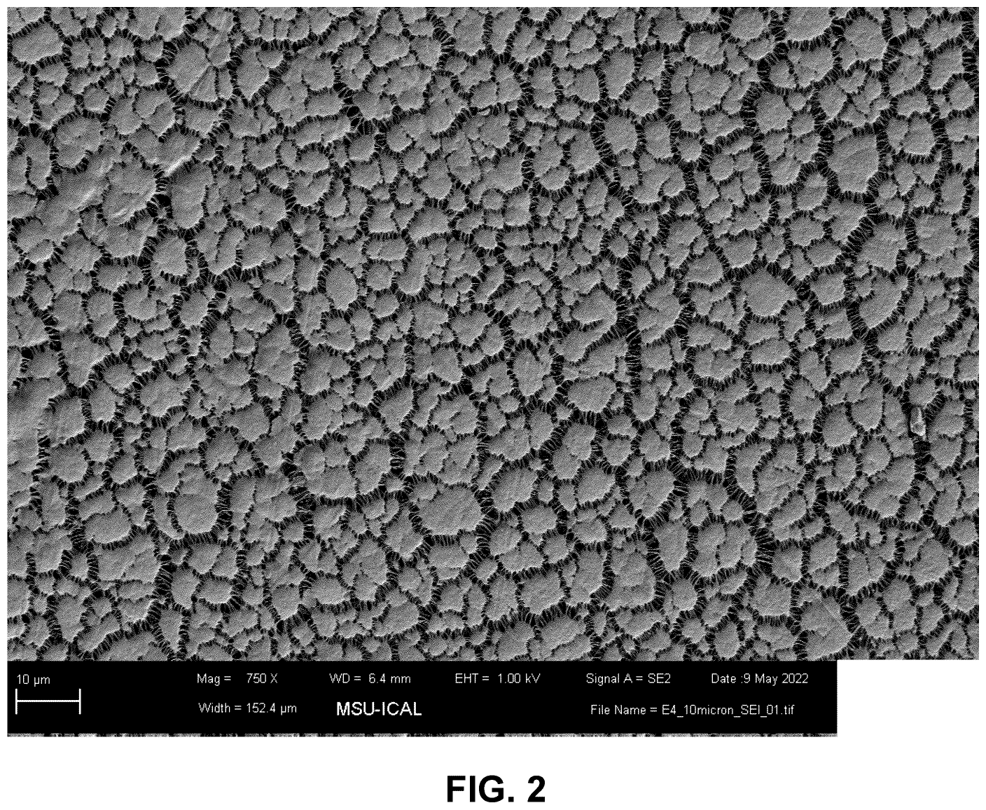

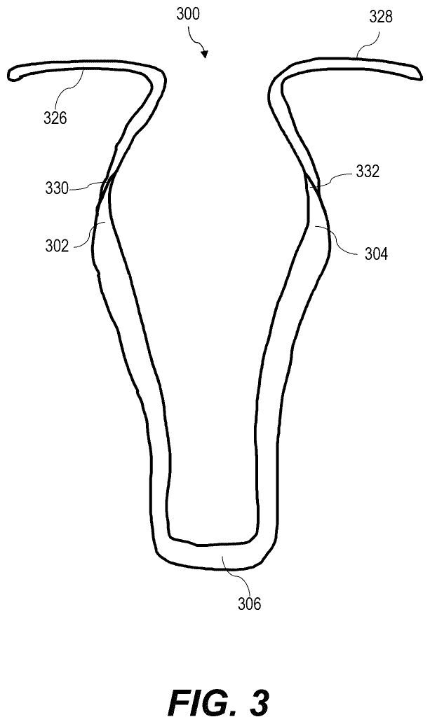

An embodiment of the ePTFE article useable as an implant device (e.g., patch) is configured as a flat (roughly/substantially planar) sheet embodiment as seen in FIGS. 3-5. The body is made of a relatively closed structure, high strength, expanded fluoropolymer, as demonstrated in FIG. 2.

The embodiments above have shown no tissue ingrowth as well as no bacterial penetration. Thus, they avoid the well-known infection problems existing in the polypropylene prior art devices. Additionally, because ingrowth is avoided, surgical removal is relatively easy to accomplish, if necessary, and leaves little, if any, scar tissue or other damage.

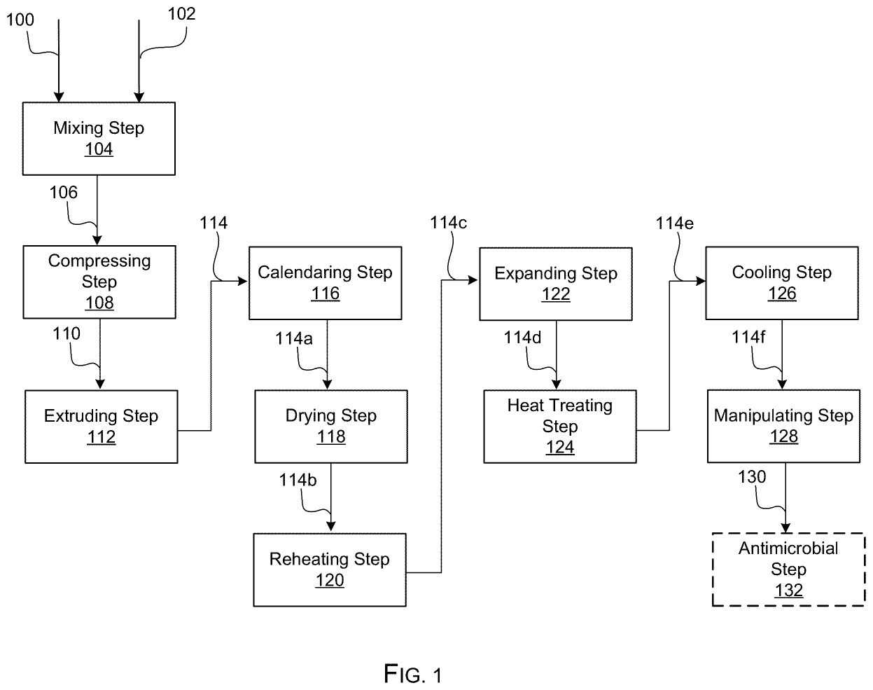

The article (e.g., articles 300, 400, and/or 500) is made, in embodiments, according to a process as shown in FIG. 1. More specifically, the article is produced by expanding PTFE in multiple dimensions (i.e., multiaxially). First, an amount of a processed fluoropolymer 100 and an amount of an extrusion-aid 102, such as mineral spirits, are mixed to form a resin paste 106 in a mixing step 104. The paste 106 is then subjected to a compressing step 108 and is compressed at relatively low pressures into a material 110 in a pre-extrusion form, e.g., as a pellet.

Next, the material 110 is extruded in an extruding step 112 into a substantially flat PTFE article 114.

The PTFE article 114 is then calendared while wet in a calendaring step 116 to a desired thickness. Because the article 114 will be partially wet with the extrusion-aid 102 (e.g. mineral spirits), the article 114a then moves on to a drying step 118 where the extrusion aid 102 is removed by subjecting the article 114a to a temperature slightly above the boiling point of the extrusion aid 102 (e.g., about 150° C.), and far below the sintering or coalescing temperature of the fluoropolymer 100, generally at about 327° C. in embodiments.

Next, the dried article 114b, in embodiments, moves on to a reheating step 120 and is reheated at a temperature higher than the drying temperature, but below the melt temperature, e.g., above 240° C., in embodiments, or about 250° C. in more specific embodiments.

Next, the article 114c is expanded during an expanding step 122 in one or more dimensions. In embodiments where the article 114c is expanded in multiple dimensions, or in other words “multiaxially expanded,” the process will result in multidirectional elongated fibrils (which are substantially parallel) extending between nodes (see FIG. 2). It is possible that embodiments could be additional dimensions of planar expansion, or even in a single axis of expansion. In the disclosed embodiment, however, the fibrils will have lengths of between about 0.5 microns to about 3.0 microns between the nodes, which creates pores between the nodes. In embodiments, the resulting pore size is about less than or equal to 3.0 by 1.0 microns. Again here, these pore sizes will not allow for tissue ingrowth, and the multiaxial expansion gives great strength. Also with the multiaxial arrangement, the extent of expansion of the PTFE makes the material softer, and here, that softness makes the ultimate device perform well.

Next, after expansion, the expanded article 114d is subjected to a final heat-treating step 124. In this step, the article 114d is restrained in its expanded state and heated above the thermal transition temperature at about 350° C. to lock the structure of the article 114d in place.

The now expanded and locked ePTFE article 114e is allowed to cool during a cooling step 126 over a period of time at a lower temperature, e.g., at ambient. This results in a final ePTFE article 114f that can be manipulated during one or more manipulating steps 128 into a surgical implant patch 130, embodiments of which are shown in FIGS. 3, 4, and 5. For example, the article 114e may be cut to form from a rectangular, square, or other shaped sheet for a more specific patch shape intended to meet surgical needs.

Once the article 114 has been processed as discussed above into the patch 130, the patch 130 can be presented for use along with existing implant systems and/or methods. The patch 130 can also be configured for use as a surgical implant.

As an optional additional step 132, an antimicrobial coating can be applied to the ePTFE patch 130.

Portions of any of the exemplary articles (article 300, 400, or 500) can further be configured for attachment to placement aids (e.g., needles, plastic tips, rubber tips, etc.)

### Example 1

In an embodiment, a resin paste was formed by blending 100% PTFE fine powder with an extrusion-aid (e.g., mineral spirits). The resulting resin paste was then formed into an extrusion pellet.

The PTFE article was extruded as a rectangular cross section, and calendared to a thinner cross section while wet. The ultimate thickness was about 0.60 mm after calendaring.

After calendaring, the lubricant was removed by subjecting the article to heat at a temperature of about 150° C. in order to dry the article (remove the mineral spirits).

With the lubricant now removed, the process moved on to an expansion step. In this embodiment of the expansion step, the article was then reheated at about 250° C. but below the melt temperature and expanded multiaxially. In an embodiment, a single expansion step occurs in which the article is secured on at least at four different sides and simultaneously expanded in first and second directions. In some embodiments, the first direction and the second direction may be offset by 90 degrees (e.g., longitudinally and laterally). In some embodiments, the expansion could occur in more than two dimensions. While the description above references only two directions as being “multiaxial,” it is to be understood than any number of expansion axes (e.g., two, three, four, five, six, or more) may be used for multiaxial expansion.

Following expansion, the material was restrained in its expanded state, and heated above the thermal transition temperature at about 350° C. to lock the structure in place.

Now expanded, the ePTFE article was allowed to cool over a period of time at a lower temperature, e.g., at ambient.

The extent of expansion of the PTFE was shown to give the material softness and other desired parameters discussed above making it ideal for use as a surgical patch.

FIG. 2 is a micrograph taken of the material manufactured according to the processes discussed in FIG. 1. Referring to the figure, it can be seen that the article has fibril lengths which, generally speaking, range from about 0.5 micron to about 1.0 micron. The multiaxial expansion creates elongated pores between the nodes which have widths that range in size, but are generally lower than 2 micron. This size inhibits cells or bacteria from penetrating the material. Again here, fibrils occupy limited space in the article, and the node sizes are relatively larger. As can be seen from the FIG. 2 micrograph, more than about 80% of the area is comprised of solid nodes.

The descriptions above relate to the use of ePTFE articles as prosthetic patches to stabilize any number of the patient's organs, such as the urethra or bladder as described above. Additionally, multiple strips could be used together for certain applications. Further, the articles could be used for the support of rectal muscles in other applications. In some instances, present embodiments may be used to treat certain forms of hernias. These include, but are not limited to, inguinal, femoral, incisional, ventral, umbilical, and hiatal.

Some embodiments of the present disclosure may be used in urogynecological procedures. These include stress urinary incontinence and pelvic organ prolapse (POP). In patients exhibiting POP, embodiments disclosed herein may be applied transvaginally or transabdominally. In embodiments, one or more anchoring aids, such as sutures could be attached to the article to aid in placement of the article. In embodiments, this includes pre-attachment of sutures prior to placement of the article.

FIG. 3 depicts an article 300 that may be used, in some embodiments, to treat POP. Specifically, FIG. 3 illustrates an anterior view of article 300 as it may be inserted into the patient experiencing POP. Article 300 may include a substantially flat elongated body 306 (e.g., a sheet) having ends 302, 304. In some embodiments, ends 302, 304 may be connected to placement aids 326, 328 at connection locations 330, 332, respectively. Placement aids 326, 328 may be made of ePTFE, rubber, or other biocompatible material suitable for aiding in the placement of article 200. In terms of forming a patch, one or more articles might be employed.

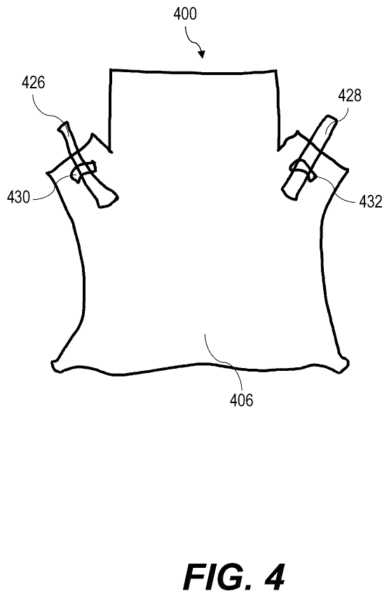

FIG. 4 depicts an article 400 that may be used, in some embodiments, to treat POP. Specifically, FIG. 4 illustrates a transverse view of article 400 as it may be placed in a patient experiencing POP. Article 400 may include a flat body 406 patch (e.g., a flat sheet) which could be used, depending on the installation, for establishing a barrier or even containment to an extent. In some embodiments, flat body 406 may be configured to span the pelvic floor of the patient for organ and/or tissue containment. Optionally, in some embodiments article 400 includes placement aids 426, 428 connected to flat body 406 at connection locations 430, 432, respectively. Placement aids 426, 428 may be made of ePTFE, rubber, or other biocompatible material suitable for aiding in the placement of article 400. Alternatively, as discussed above, the placement aids 426, 428 may comprise one or more sutures.

Articles 300, 400 are particularly well suited for treatment of urogynecological disorders, such as POP. In POP, the pelvic floor no longer supports pelvic organs, including the vagina, cervix, uterus, bladder, urethra, and rectum. Accordingly, providing support to the pelvic floor using article 300 or 400 decreases the chances of adverse effects (e.g., scarring due to insertion), decreases the risk of bacterial infection, and extends the life of the article/patch.

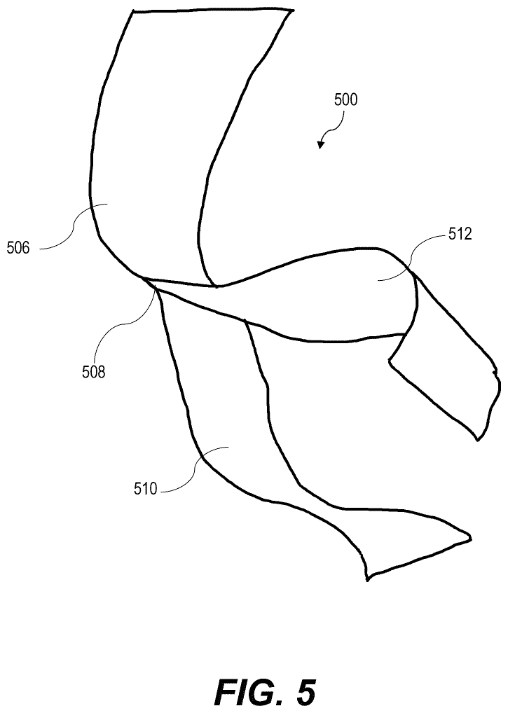

FIG. 5 depicts an article 500 that may be used, in some embodiments, to treat POP. Article 500 may similarly include a substantially flat elongated body 506. Differing from other embodiments, article 500 may include a divergence point 508 whereby the substantially flat elongated body 506 splits into a first prong 510 and a second prong 512. In these embodiments, each extension of article 500 may be configured to support multiple compartments. In a non-limiting example, the substantially flat elongated body 506 may support the bladder compartment, the first prong 510 may support the vaginal canal, and the second prong 512 may support the rectocele compartment. However, it is envisioned that each extension of article 500 may support tissues other than those mentioned above. Accordingly, article 500 is particularly advantageous for treatment of POP since multiple tissues in the pelvic floor area may be simultaneously supported by article 500.

As discussed above, in embodiments articles 300, 400, 500 are substantially formed of a fluoropolymer. In some embodiments, articles 300, 400, 500 are substantially formed of PTFE. In more specific embodiments, articles 300, 400, 500 are substantially formed of expanded PTFE (ePTFE). Additionally, articles 300, 400, 500 may be multiaxially expanded, as described above.

Features described above as well as those claimed below may be combined in various ways without departing from the scope hereof. The following examples illustrate some possible, non-limiting combinations:
(A1) A process of making a surgical implant configured to provide support for a tissue following pelvic organ prolapse, the process including: selecting an amount of a processed fluoropolymer; creating a paste by combining the amount of the processed fluoropolymer with an extrusion aid; compressing the paste into a pre-extrusion form to create a material; extruding the material to create an article that is substantially flat; calendaring the article to a desired thickness; drying the article by applying heat to the article at a first temperature; expanding the article in one or more directions; heat treating the article while the article is restrained in an expanded state to lock a structure of the article in place; and allowing the article to cool to result in an expanded fluoropolymer article; manipulating the expanded fluoropolymer article into a patch body, the patch body is for use as the surgical implant.
(A2) For the process denoted as (A1), the manipulating the expanded fluoropolymer article into the patch body includes cutting the expanded fluoropolymer into a pre-selected shape.
(A3) For the process denoted as (A1) or (A2), the manipulating the expanded fluoropolymer article into the patch body includes forming a substantially flat elongated body that extends from a first end to a second end.
(A4) For the process denoted in any of (A1) through (A3) the manipulating the expanded fluoropolymer article into the patch body includes connecting a first placement aid to the first end and a second placement aid to the second end, the first and second placement aids are to aid in surgical placement of the patch body.
(A5) For the process denoted in any of (A1) through (A4) the manipulating the expanded fluoropolymer article into the patch body includes forming a substantially flat body with one or more placement aids connected to the substantially flat body at one or more connection locations.
(A6) For the process denoted in any of (A1) through (A5) the manipulating the expanded fluoropolymer article into the patch body includes forming a substantially flat elongated body having a divergence point wherein the substantially flat elongated body splits into a first prong and a second prong.
(A7) For the process denoted in any of (A1) through (A6) where the first temperature is above a boiling point of the extrusion aid and below a sintering and coalescing temperature of the processed fluoropolymer.
(A8) For the process denoted in any of (A1) through (A7) the expanding the article in one or more directions includes multiaxial expanding of the article such that a resulting expanded article includes a plurality of multidirectional elongated fibrils extending between a plurality of nodes such that a plurality of pores are created between the plurality of nodes.
(A9) For the process denoted in any of (A1) through (A8) where each of the plurality of multidirectional elongated fibrils has a length between approximately 0.5 microns to approximately 3.0 microns.
(A10) For the process denoted in any of (A1) through (A9) where each of the plurality of multidirectional elongated fibrils has a length between approximately 0.5 microns to approximately 1.0 micron.
(A11) For the process denoted in any of (A1) through (A10) where each of the plurality of pores has a size of less than or equal to 3.0 microns by 1.0 micron.
(A12) For the process denoted in any of (A1) through (A11) where each of the plurality pores has a size of less than or equal to 2.0 microns.
(A13) For the process denoted in any of (A1) through (A12) where the plurality of nodes form approximately 80% of a total surface area of the resulting expanded article.
(B1) A surgical implant to provide support for a tissue following pelvic organ prolapse, the surgical implant includes: a patch body formed from an expanded fluoropolymer article; the expanded fluoropolymer article comprises a plurality of nodes and a plurality of elongated fibrils, the plurality of elongated fibrils interconnecting the plurality of nodes and forming a plurality of pores between the plurality of nodes.
(B2) For the surgical implant denoted as (B1) where each of the plurality of multidirectional elongated fibrils has a length between approximately 0.5 microns to approximately 3.0 microns.
(B3) For the surgical implant as denoted in (B1) or (B2) where each of the plurality of multidirectional elongated fibrils has a length between approximately 0.5 microns to approximately 1.0 micron.
(B4) For the surgical implant as denoted in any of (B1) through (B3) where each of the plurality of pores has a size of less than or equal to 3.0 microns by 1.0 micron.
(B5) For the surgical implant as denoted in any of (B1) through (B4) where each of the plurality of pores has a size of less than or equal to 2.0 microns.
(B6) For the surgical implant as denoted in any of (B1) through (B5) where the plurality of nodes form approximately 80% of a total surface area of the patch body.
(B7) For the surgical implant as denoted in any of (B1) through (B6) where each of the plurality of nodes has a density of between approximately 2.0 grams to approximately 2.2 grams per cubic centimeter.
(B8) For the surgical implant as denoted in any of (B1) through (B7) where the expanded fluoropolymer article has a density of less than approximately 2.0 grams per cubic centimeter.

Many different arrangements of the various components depicted, as well as components not shown, are possible without departing from the spirit and scope of what is claimed herein. Embodiments have been described with the intent to be illustrative rather than restrictive. Alternative embodiments will become apparent to those skilled in the art that do not depart from what is disclosed. A skilled artisan may develop alternative means of implementing the aforementioned improvements without departing from what is claimed.

It will be understood that certain features and sub-combinations are of utility and may be employed without reference to other features and sub-combinations and are contemplated within the scope of the claims. Not all steps listed in the various figures need be carried out in the specific order described.

1. A process of making a surgical implant configured to provide support for a tissue following pelvic organ prolapse, the process comprising:
selecting an amount of a processed fluoropolymer;
creating a paste by combining the amount of the processed fluoropolymer with an extrusion aid;
compressing the paste into a pre-extrusion form to create a material;
extruding the material to create an article that is substantially flat;
calendaring the article to a desired thickness;
drying the article by applying heat to the article at a first temperature;
expanding the article in one or more directions;
heat treating the article while the article is restrained in an expanded state to lock a structure of the article in place;
allowing the article to cool to result in an expanded fluoropolymer article; and
manipulating the expanded fluoropolymer article into a patch body, the patch body configured for use as the surgical implant.

2. The process of claim 1, wherein manipulating the expanded fluoropolymer article into the patch body comprises cutting the expanded fluoropolymer into a pre-selected shape.

3. The process of claim 1, wherein manipulating the expanded fluoropolymer article into the patch body comprises forming a substantially flat elongated body that extends from a first end to a second end.

4. The process of claim 3, wherein manipulating the expanded fluoropolymer article into the patch body comprises connecting a first placement aid to the first end and a second placement aid to the second end, the first and second placement aids configured to aid in surgical placement of the patch body.

5. The process of claim 1, wherein manipulating the expanded fluoropolymer article into the patch body comprises forming a substantially flat body with one or more placement aids connected to the substantially flat body at one or more connection locations.

6. The process of claim 1, wherein manipulating the expanded fluoropolymer article into the patch body comprises forming a substantially flat elongated body having a divergence point wherein the substantially flat elongated body splits into a first prong and a second prong.

7. The process of claim 1, wherein the first temperature is above a boiling point of the extrusion aid and below a sintering and coalescing temperature of the processed fluoropolymer.

8. The process of claim 1, wherein expanding the article in one or more directions comprises multiaxial expanding of the article such that a resulting expanded article includes a plurality of multidirectional elongated fibrils extending between a plurality of nodes such that a plurality of pores are created between the plurality of nodes.

9. The process of claim 8, wherein each of the plurality of multidirectional elongated fibrils has a length between approximately 0.5 microns to approximately 3.0 microns.

10. The process of claim 8, wherein each of the plurality of multidirectional elongated fibrils has a length between approximately 0.5 microns to approximately 1.0 micron.

11. The process of claim 8, wherein each of the plurality of pores has a size of less than or equal to 3.0 microns by 1.0 micron.

12. The process of claim 8, wherein each of the plurality pores has a size of less than or equal to 2.0 microns.

13. The process of claim 8, wherein the plurality of nodes form approximately 80% of a total surface area of the resulting expanded article.

14. A surgical implant configured to provide support for a tissue following pelvic organ prolapse, the surgical implant comprising:
a patch body formed from an expanded fluoropolymer article;
wherein the expanded fluoropolymer article comprises a plurality of nodes and a plurality of elongated fibrils, the plurality of elongated fibrils interconnecting the plurality of nodes and forming a plurality of pores between the plurality of nodes.

15. The surgical implant of claim 14, wherein each of the plurality of multidirectional elongated fibrils has a length between approximately 0.5 microns to approximately 3.0 microns.

16. The surgical implant of claim 14, wherein each of the plurality of multidirectional elongated fibrils has a length between approximately 0.5 microns to approximately 1.0 micron.

17. The surgical implant of claim 14, wherein each of the plurality of pores has a size of less than or equal to 3.0 microns by 1.0 micron.

18. The surgical implant of claim 14, wherein each of the plurality of pores has a size of less than or equal to 2.0 microns

19. The surgical implant of claim 14, wherein the plurality of nodes form approximately 80% of a total surface area of the patch body.

20. The surgical implant of claim 14, wherein each of the plurality of nodes has a density of between approximately 2.0 grams to approximately 2.2 grams per cubic centimeter.

21. The surgical implant of claim 14, wherein the expanded fluoropolymer article has a density of less than approximately 2.0 grams per cubic centimeter.
